(19) United States
(12) Patent Application Publication
(10) Pub. No.: US 2016/0035091 A1
(43) Pub. Date: Feb. 4, 2016

(54) Methods and Apparatus for Quantifying Inflammation

(71) Applicant: IMAGE ANALYSIS LIMITED, London (GB)

(72) Inventor: Olga Kubassova, London (GB)

(73) Assignee: Image Analysis Limited, London (GB)

(21) Appl. No.: 14/783,397

(22) PCT Filed: Apr. 9, 2014
(86) PCT No.: PCT/GB2014/051107

§ 371 Date: Oct. 8, 2015

(30) Foreign Application Priority Data
Apr. 9, 2013 (GB) 1306434.0

(51) Int. Cl. G06T 7/00; A61B 5/00; G06K 9/42; A61B 5/055; G06T 15/08; G06K 9/62

(52) U.S. Cl. CPC G06T 7/0012; G06T 15/08; G06K 9/6267; G06K 9/42; A61B 5/055; A61B 5/41; G06T 2200/04; G06T 2207/10132; G06T 2207/10088; G06T 2207/10081; G06T 2207/30024; G06T 2207/30196; G06T 2207/20104

(57) ABSTRACT

A computer-implemented method and apparatus for quantifying inflammation in tissue or anatomy. The method includes analysing Dynamic Contrast Enhanced MRI data. The analysis comprises determining a value quantifying inflammation in the tissue. The value is a continuous score value and small changes in the inflammation result in a change in the determined value.

### FIELD OF THE INVENTION

The present invention relates to analysing image data for quantifying inflammation in tissue or anatomy.

### BACKGROUND OF THE INVENTION

In rheumatoid arthritis (RA) and other inflammatory conditions, early diagnosis combined with early initiation of appropriate therapy is important for improved clinical outcomes. Magnetic Resonance Imaging (MRI) provides a good contrast between different soft tissues of the body. MRI image data can be studied to provide an indication of inflammation in tissue or anatomy.

Dynamic Contrast Enhanced Magnetic Resonance Imaging (DCE-MRI), which is a sequence of MRI, involves the acquisition of sequential images in rapid succession every few seconds during and after an intravenous administration of contrast agent. The contrast agent improves the visibility of internal body structures and different tissue types can be distinguished depending on the temporal pattern of uptake of the contrast agent.

Starting from a baseline, perfused or highly vascular tissues absorb the contrast agent, and their signal intensity increases. This increasing phase is termed the wash-in phase. The intensity usually increases up to a certain value and then exhibits a plateau (of variable width) followed by wash-out phase in which the signal intensity gradually decreases.

The blood vessels in highly vascular tissue, such as tissue exhibiting active synovitis, can exhibit fast uptake, e.g. steep wash-in, and retain the contrast for a short time when equilibrium is reached between the smaller blood vessels and the extracellular phase (showing plateau) and then release the contrast media, back into the blood stream again, which corresponds to the wash-out phase.

If the tissue does not take up any contrast agent, no enhancement pattern or changes in intensity will be observed over time. The signal intensity versus time curve will be constant with variations attributable to the noise due to hardware instability or patient movement. If the tissue had never taken up enough contrast agent to plateau, then only the baseline and wash-in phases will be present.

Prior art methods for analysing MRI data involve manual outlining of regions, the volume or area of which is measured by an observer. This process is time consuming and the reproducibility of such methods is extremely low and highly subjective to the observer's experience.

Other prior art methods quantify inflammation by providing a score value, wherein the score value is a discrete value from a limited range of possible values. One MRI scoring system for disease activity in rheumatoid arthritis is the Rheumatoid Arthritis Magnetic Resonance Imaging Score (RAMRIS) system, as disclosed for example in the paper “OMERACT Rheumatoid Arthritis Magnetic Resonance Imaging Studies. Exercise 5: an international multicentre reliability study using computerised MRI erosion volume measurements” from the Journal of Rheumatology, Vol. 30, no. 6., pp 1380-4. 2003, by P. Bird, B. Ejbjerg, F. McQueen, M. Ostergaard, M. Lassere and J. Edmonds.

In RAMRIS, the scoring of synovial and bone marrow changes is done in the wrist and the second to the fifth MCP joint on a discrete scale of integers from 0 to 3 for every examined joint area, where a score of 0 corresponds to a normal joint, and scores 1, 2 and 3 reflect mild, moderate, and severe disease activity respectively. In addition, but separately, erosions are scored in the same areas from 0-10 in every bone where score 0 correspond to no erosions, score 1 corresponds to 1-10% of the involved bone, score 2 corresponds to 11-20% of the involved bone etc. up to 100%. Erosion volume in the long bones such as the distal radius/Ulna and the MCP and inter-phalangeal bones are scores using an imaginary cut-off 1cm from the joint surface. This scoring system has proven to be reliable and reproducible in the hands of calibrated and skilled users, but needs extensive training and is relative time-consuming preventing it being applied in daily clinical practice.

Existing scoring systems such as RAMRIS have a very limited number of output values. The output of existing scoring systems is discontinuous since small changes in the inflammation will either not result in a change in the output score or will result in a jump in the output score, say from 1 to 2. This means that the RAMRIS scoring system does not provide a score which allows a clinician to distinguish precisely between variations in inflammation.

### SUMMARY OF THE INVENTION

In a first aspect of this disclosure, there is provided a computer-implemented method for quantifying inflammation in tissue or anatomy, comprising: acquiring image data pertaining to at least one image of tissue or anatomy; analysing the image data comprising determining a first value quantifying inflammation in the tissue or anatomy; and outputting said first value, wherein the first value is determined to be a continuous score value residing in a range of continuous score values quantifying the inflammation.

The first value is a continuous score value residing in a range of continuous score values quantifying the inflammation. The output value is continuous since any change in the inflammation will always result in a corresponding change in the output value. The resolution of the scoring is much greater than in previous scoring systems. The present invention extracts continuous real numbers, for example to measure the aggressiveness of the inflammation and/or the volume of inflammation. This is in contrast to the more subjective discrete and less precise scoring for rheumatoid arthritis patients using RAMRIS or other semi quantitative scoring methods.

The scoring methodology of the present invention advantageously provides an objective, sensitive and repeatable quantification of inflammation driven changes, crucial for accurate assessment of therapeutic response and desirable for early disease detection. The scoring mechanism is advantageously simple to use, adequate to detect disease related changes, and effective in guiding treatment strategy.

The method is advantageously robust, repeatable and gives an objective score. Furthermore, the method is versatile and is able to consistently and with high repeatability quantify inflammatory changes in various joints and is not dependent on any particular anatomy. The method is sensitive and able to measure even subtle changes in disease activity. The method is able to quantify inflammatory changes in images acquired with scanners manufactured by various vendors and of different strength, e.g. Tesla measurement in MRI.

The computer support enables a reader to achieve highly reproducible results obtained in a fully-automated manner or when a clinical professional is involved for some of the analysis and the scores are obtained in a semi-automate manner.

The step of acquiring image data may comprise acquiring or inputting image data to a processor.

The step of acquiring image data may comprise extracting data from a memory and inputting it into the processor. The image data may contain pixel values of signal intensity output from a scan over a scanned area. The image data may be stored in an image file containing these pixel values, for example in a bitmap file, JPEG or other image file format containing image pixel values representative of signal intensity.

The step of analysing image data may involve analysing the image data with a processor.

The step of outputting said first value may involve outputting said first value with a processor.

The term “processor” may include one or more discrete processing units which are coupled to each other within one or more electronic circuits. The processing units may be integrated on the same electronic circuit or connected to each other across multiple electronic circuits, e.g. over a network to perform the individual steps of the method or underlying processing substeps.

The step of analysing may comprise determining the first value based on a continuous function being applied to the image data.

The first value may quantify the volume of inflammation.

The step of analysing the image data may further comprise determining the first value by quantifying the volume of inflammatory activity in the tissue or anatomy.

The step of quantifying the volume of inflammatory activity may comprise quantifying based on a first continuous function being applied to the image data.

The first value may quantify the aggressiveness of inflammatory activity. The computer-implemented method may determine and output a first value and a second value, wherein the first value quantifies the aggressiveness of the inflammatory activity and the second value quantifies the volume of inflammatory activity.

The step of analysing the image data may further comprise determining the first value by quantifying the aggressiveness of inflammatory activity in the tissue or anatomy.

The step of quantifying the aggressiveness may comprise quantifying based on a second continuous function being applied to the image data.

Each image may be a magnetic resonance image (MRI). The MRI image is obtained by an MRI scan.

The image may be a plurality of temporal magnetic resonance images. These images may have been obtained by DCE-MRI.

Alternatively, each image may be a computed axial tomography image or an ultrasound image.

The tissue may have been exposed to a contrast agent.

Analysing the data may comprise analysing a temporal pattern of contrast agent uptake.

The method may further comprise identifying a region of interest of the tissue and selectively analysing data pertaining to the image of the tissue or anatomy in the region of interest. Alternatively, the entire image may be analysed. The selected region of interest may be selected by a user using an input device for example a computer pointing device.

The analysed data may comprise signal intensity values for one or more pixels of the image at one or more time points. The signal intensity value of each pixel may be variable. A pixel may include one or more discrete identified values of the image. A pixel may convey a single measured signal intensity value or it may convey an average of at least two measured signal intensity values.

The measured signal may provide one or more measured signal intensities as determined from an image acquisition device, such as an MRI scanner. The signal intensity value for one or more pixels may comprise multiple components, e.g. red, green and blue (RGB) or cyan, magenta, yellow, and key (black) (CMYK), each component having a magnitude such that the overall signal intensity value corresponds to a particular colour or greyscale which represents the measured signal, following processing by a processor of the measured signal.

Analysing the data may comprise classifying one or more pixels of the image into groups, each group representative of a tissue type.

Analysing the data may comprise analysing a temporal pattern of contrast agent uptake for said one or more pixels of the image for determining the tissue type of each pixel.

Analysing the data may comprise identifying one or more pixels of the image of a first tissue type.

Analysing the data may comprise summing the number of pixels of the image determined to be of the first tissue type.

Analysing the data may comprise identifying one or more pixels of the image of a second tissue type.

Analysing the data may comprise summing the number of pixels of the image determined to be of the second tissue type.

Analysing the data may comprise normalizing the number of pixels of the first tissue type and/or of the second tissue type.

The normalization may be based on the dimensions of the imaged tissue, or number of pixels in the image as a whole. Normalizing may comprise determining a mean baseline signal intensity value. Normalizing may further comprise subtracting the mean baseline signal intensity value from the signal intensity values. Normalizing may further comprise dividing the resulting values by the mean baseline value to express the signal intensity values in terms of multiples of the mean baseline value. Normalization may involve performing the following calculation:

$$Normalized signal intensity value = \frac{( SImeasured - baseline value )}{baseline value}$$

whereby SI measured corresponds to or is a measured signal intensity value; and baseline value corresponds to or is a baseline signal intensity value in particular a mean baseline signal intensity value.

The first value may be a function of the total number of pixels of the first tissue type and/or the second tissue type.

The first value may be a function of the normalized number of pixels of the first tissue type and/or of the normalized number of pixels of the second tissue type.

The first tissue type may be tissue identified as having a plateau enhancement.

The first tissue type may be tissue identified as having a wash-out enhancement.

The second tissue type is tissue identified as having a plateau enhancement. For example, determining the first value may comprise determining DEMRIS(Volume) according to:

$$DEMRIS ( Volume ) = {\sum }_{area of interest} N\_plateau + N\_washout$$

whereby DEMRIS(Volume) is the sum of N_plateau and N_washout in the area of interest, wherein the area of interest is the entire image or a selected region of interest. DEMRIS(Volume) may also be referred to as DEMRIQ(Volume). DEMRIQ stands for Dynamic contrast Enhanced MRI Quantification.

N_plateau corresponds to or is the total number of pixels with a plateau pattern of enhancement in the area of interest; and N_washout corresponds to or is the total number of pixels with washout pattern of enhancement in the area of interest.

N_plateau and N_washout may have been normalized to the area of the joint or the physical size.

The output value may be DEMRIS(Volume), or a value based on or corresponding to DEMRIS(Volume), e.g. a function of DEMRIS(Volume), for example DEMRIS(Volume) x a constant, e.g. 2, 5, 10, 50, 100 or 1000.

The method may further comprise generating display data for display pertaining to a parametric map, preferably a colour-coded parametric map, for an observer to visualise locations of different tissue types. The method may further comprise generating display data for display pertaining to a map of the maximum enhancement and/or of the initial rate of enhancement for each pixel. The method may further comprise displaying the display data on a display.

The method may further comprise selecting a region of interest of the tissue based on input from the observer. The user provides input via an input device, for example a computer pointing device.

Analysing the data may comprise determining an initial rate of enhancement in an inflamed area.

Analysing the data may further comprise determining an initial rate of enhancement in a blood vessel.

The first value may be a function of the initial rate of enhancement in the inflamed area.

The output value may be a function of the initial rate of enhancement in the inflamed area and the initial rate of enhancement in the blood vessel.

The output value may be a function of the ratio of the initial rate of enhancement in the inflamed area and the initial rate of enhancement in the blood vessel.

Analysing the data may comprise determining a mean initial rate of enhancement (IRE) in an inflamed area. An inflamed area may be an area which has been identified as tissue and which exhibits at least a contrast uptake phase and a plateau phase.

Analysing the data may comprise determining a mean initial rate of enhancement (IRE) in the blood vessel. Analysing the data may comprise identifying a number of pixels in an image corresponding to one or more blood vessels. Each pixel has its own sequence of intensity values over time. Determining an average initial rate of enhancement for a blood vessel reduces the effects of outside factors on the intensity values, including inequalities in blood contrast concentration, partial volume effects, MRI flow artifacts, scanner noise and patient motion.

For example, determining the first value may comprise determining DEMRIS(Inflammation) according to:

$$DEMRIS ( Inflammation ) = \frac{mean ( IRE inflamed area )}{mean ( IRE blood vessel )}$$

whereby mean (IRE inflamed area) is or corresponds to the mean initial rate of enhancement in the inflamed area and mean (IRE blood vessel) is or corresponds to the mean initial rate of enhancement in a blood vessel.

Alternatively, DEMRIS(Inflammation) may be a combination of DEMRIS(Volume) multiplied by the mean initial rate of enhancement in the area of interest (mean (IRE)) or multiplied by the mean maximum enhancement in the area of interest (mean (ME))

$$DEMRIS ( Inflammation ) = mean ( IRE ) * {\sum }_{area of interest} Nplateau + Nwashout$$

$$or$$

$$DEMRIS ( Inflammation ) = mean ( ME ) * {\sum }_{area of interest} Nplateau + Nwashout$$

The area of interest may be the region of interest or the entire image.

DEMRIS(Inflammation) may also be referred to as DEMRIQ(Inflammation).

The output value may be DEMRIS(Inflammation) or a value based on or corresponding to DEMRIS(Inflammation), e.g. a function of DEMRIS(Inflammation), for example DEMRIS(Inflammation)×a constant, e.g. 2, 5, 10, 50, 100 or 1000.

Determining the first value may comprise determining a value quantifying the volume of inflammation in the region of interest, such as DEMRIS(Volume), and the method may further comprise determining a second value quantifying the aggressiveness of inflammation in the region of interest, wherein the second value is determined to be a continuous score value residing in a range of continuous score values quantifying the inflammation. The second value may be DEMRIS(Inflammation).

The first and second values may be output separately, or combined to provide a single output value.

The output value may be a function of the ratio of the mean initial rate of enhancement in the inflamed area and the mean initial rate of enhancement in the blood vessel.

Analysing the initial rate of enhancement in the inflamed area and/or in the blood vessel may comprise measuring the slope of signal intensity curves for one or more pixels of the image.

Analysing the data may comprise approximating the slope of each curve by a linear segment.

The method according may further comprise correcting the images for patient movement.

Analysing the data may comprise normalizing the signal intensity curves of one or more pixels of the image to a baseline, preferably by subtracting the mean values of pre-contrast frames from all other planes.

The first value may be any real value in a continuous range of values between 0 and 1; 0 and 5; 0 and 10; 0 and 100; or 0 and 1000.

The method may be for quantifying inflammation in tissue or anatomy of patients with inflammatory arthritis. The scoring method can be used to quantify inflammation in, for example, rheumatoid arthritis, psoriatic arthritis, lupus, ankylosing spondylitis or osteoarthritis, and any other inflammatory or immune driven musculoskeletal conditions.

The method may be for quantifying inflammation in tissue, for example in tissue of inflammatory joint diseases such as rheumatoid arthritis, gout, psoriatic arthritis and degenerative diseases such as osteoarthritis or anatomy of patients with cancer, for example breast, prostate or liver cancer. The method may be for quantifying inflammatory lesions such as in brain cancer, multiple sclerosis, Alzheimer's disease or dementia. The method may be for quantifying perfusion in cardio-vascular conditions such as myocardium perfusion.

Outputting said first value may comprise conveying said first value to a user.

Analysing the image data may comprise correcting the image data for patient motion. Motion correction can be done for two dimensional frames or three dimensional volumes. Alignment may be achieved by rotating and translating frames, for example by shifting frames. Alignment may be achieved by skewing frames. Rigid and non-rigid algorithms may be applied for patient motion correction.

In a second aspect of the disclosure, there is provided a computer program comprising executable instructions for execution on a computer, wherein the executable instructions are executable to perform the method described herein.

In a third aspect of the disclosure, there is provided an apparatus for quantifying inflammation in tissue, comprising: a memory, wherein the memory comprises the computer program defined above; and a processor for executing the computer program.

The apparatus may comprise a display for displaying or representing said first value when output.

In a fourth aspect of the disclosure, there is provided an apparatus configured to perform the method described herein. The apparatus may comprise one or more processors for performing the steps of the method, and optionally a memory for storing data and/or values being processed and/or output. The apparatus may comprise a display for displaying or representing said first value when output.

### BRIEF DESCRIPTION OF THE DRAWINGS

The foregoing and other objects, features and advantages of the invention will be apparent from the following description and drawings, in which:

FIG. 1 is a flowchart illustrating a method in accordance with the present invention;

FIG. 2 is a flowchart illustrating a method in accordance with the present invention;

FIG. 3 is a flowchart illustrating a method in accordance with the present invention;

FIG. 4 is a representation of the decomposition of a three dimensional MRI scan into slices at two different time points;

FIG. 5 is an example of a signal intensity versus time curve for tissue which did not absorb contrast agent;

FIG. 6 is an example of a signal intensity versus time curve for tissue with a persistent pattern of enhancement;

FIG. 7 is an example of a signal intensity versus time curve for tissue with a plateau pattern of enhancement;

FIG. 8 is an example of a signal intensity versus time curve for tissue with a washout pattern of enhancement;

FIG. 9 is a contrast uptake map in 3D where each voxel is colour coded according to the pattern of enhancement, whereby tissue having a persistent pattern of enhancement is shown in blue and is indicated by reference “C”, tissue having a plateau pattern of enhancement is shown in green and is indicated by reference “A” and tissue having a washout pattern of enhancement is shown in red and is indicated by reference “B”;

FIG. 10 shows a sample signal intensity graph;

FIG. 11 shows a normalized version of the graph shown in FIG. 10;

FIG. 12 illustrates a map of contrast uptake for a single temporal slice, whereby tissue having a persistent pattern of enhancement is shown in blue and is indicated by reference “C”, tissue having a plateau pattern of enhancement is shown in green and is indicated by reference “A” and tissue having a washout pattern of enhancement is shown in red and is indicated by reference “B”;

FIG. 13 illustrates a map of maximum enhancement (ME) for a single temporal slice, whereby tissue having a first value for the maximum enhancement is shown in yellow and is indicated by reference “D” and tissue having a second value for the maximum enhancement is shown in red and is indicated by reference “E”, wherein the first value is greater than the second value;

FIG. 14 illustrates a map of initial rate of enhancement (IRE) for a single temporal slice whereby tissue having a first value for the IRE is shown in yellow and is indicated by reference “G” and tissue having a second value for the IRE is shown in red and is indicated by reference “F”, wherein the first value is greater than the second value;

FIG. 15 illustrates a map of time of onset (T_onset) of enhancement for a single temporal slice whereby tissue having a first value for T_onset is shown in yellow and is indicated by reference “I” and tissue having a second value for T_onset is shown in red and is indicated by reference “H”, wherein the first value is greater than the second value;

FIG. 16 illustrates a map of initial rate of washout (IRW) for a single temporal slice whereby tissue having a first value for IRW is shown in blue and is indicated by reference “J”, tissue having a second value for IRW is shown in yellow and is indicated by reference “L” and tissue having a third value for IRW is shown in red and is indicated by reference “K”, wherein the first value is greater than the second value and the second value is greater than the third value;

FIG. 17 illustrates a map of time of washout (T_washout) for a single temporal slice whereby tissue having a first value for T_washout is shown in red and is indicated by reference “M”;

FIG. 18 shows four DCE-MRI frames with highly visible patient motion which are superimposed on top of each other; the outer boundaries of the wrist are outlined in red and are indicated by reference “N” to show the range of motion;

FIG. 19 relates to FIG. 18 and shows the result of ‘subtraction’ of frame 1 from frame 3 to demonstrate the range of movement;

FIG. 20 illustrates motion correction; and

FIG. 21 illustrates a system for performing the method described herein.

### DETAILED DESCRIPTION

In the following sections detailed descriptions of embodiments of the invention are given. The description of both preferred and alternative embodiments though thorough are exemplary embodiments only, and it is understood that variations, modifications and alterations may be apparent. It is therefore to be understood that said exemplary embodiments do not limit the broadness of aspects of the underlying invention.

A patient is imaged for example using DCE-MRI, with Gadolinium as a contrast agent. An MRI image is three dimensional and can be viewed in sequential planes or slices 401, as illustrated in FIG. 4. Each slice is composed of a number of pixels. The image data comprises signal intensity values for each of the pixels or for a group of one or more pixels, sampled at a number of different time points. In DCE-MRI, images are obtained at multiple time points. FIG. 4 illustrates just two of these time points at t=0 s and t=300 s.

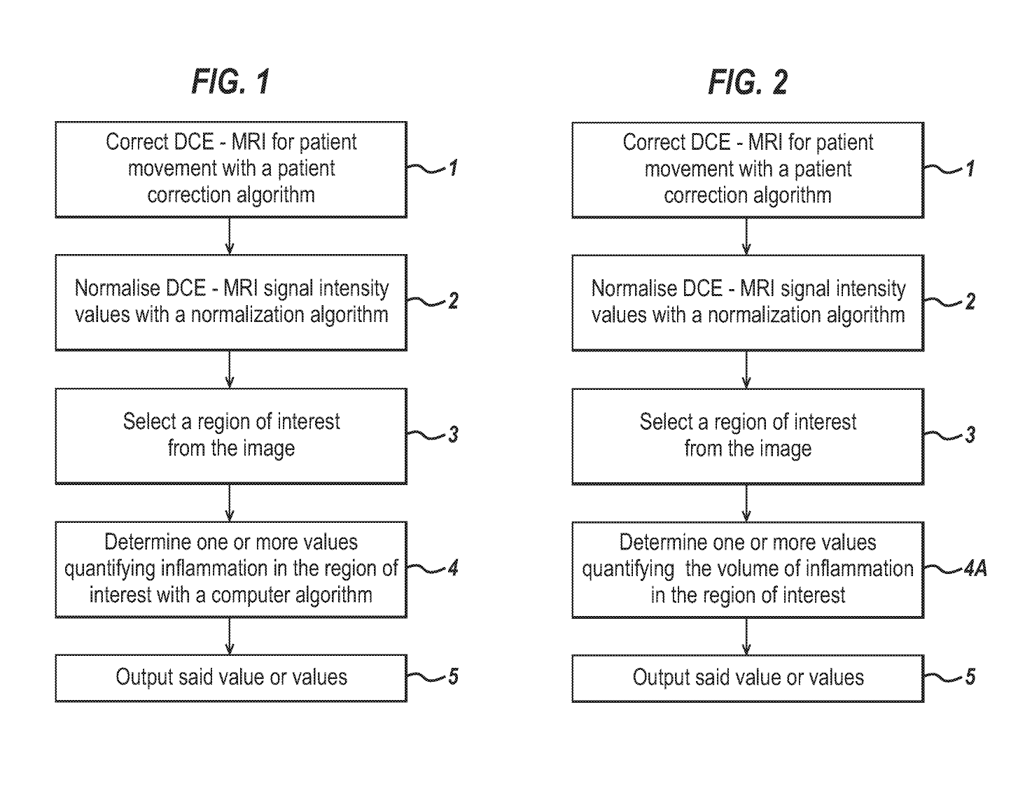

The image data is analysed by a computer to determine values which quantify inflammation in the tissue or anatomy. FIG. 1 outlines steps 1 to 5 in which firstly the DCE-MRI images are corrected for patient movement with a patient correction algorithm in step 1. The image data includes signal intensity (SI) values for each pixel at a number of time points. The signal intensity values are normalized with a normalization algorithm in step 2. A region of interest on the image is selected in step 3. The region of interest may be the entire image. One or more values quantifying inflammation in the region of interest are determined with a computer algorithm in step 4. In another embodiment (not illustrated), steps 1 to 4 are carried out in a different order. In step 5, a value is output and conveyed to a user. The value quantifies inflammation and can be used by the user directly to determine the severity of the inflammation or to compare the value with a previous value for the patient to determine changes in the inflammation.

The value is a score value which may quantify the volume or the aggressiveness of inflammatory activity. The value is determined through computer-aided detection and quantification of inflammatory activity.

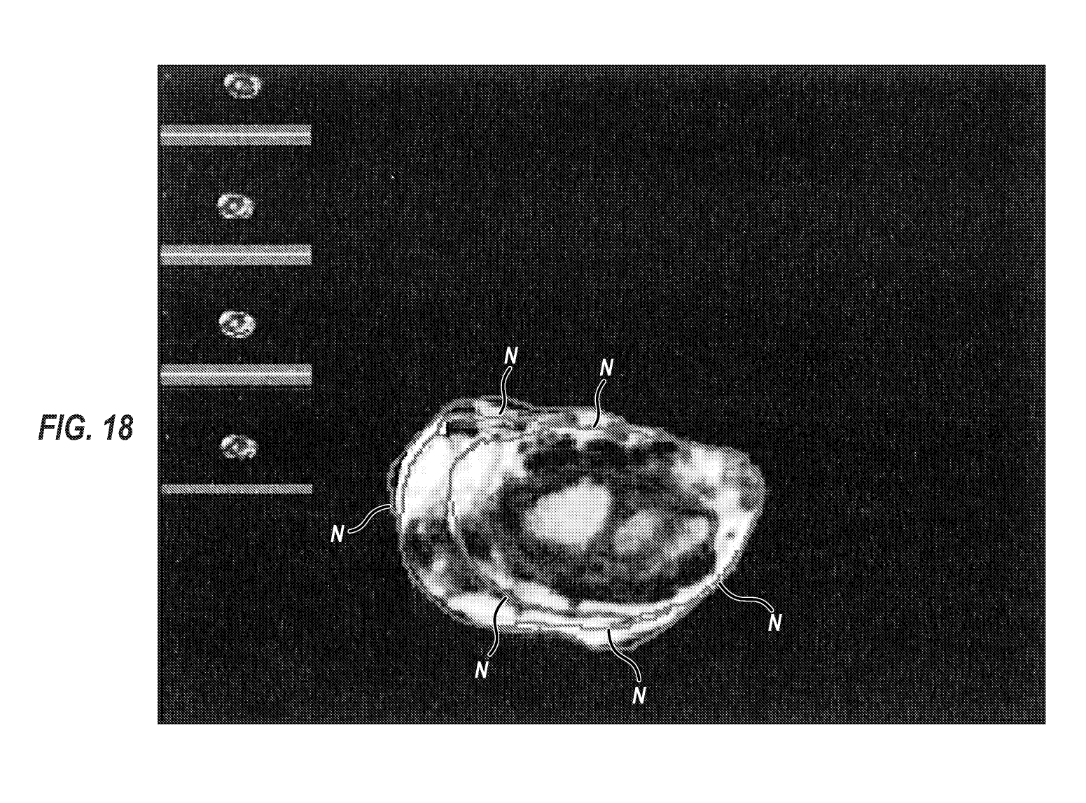

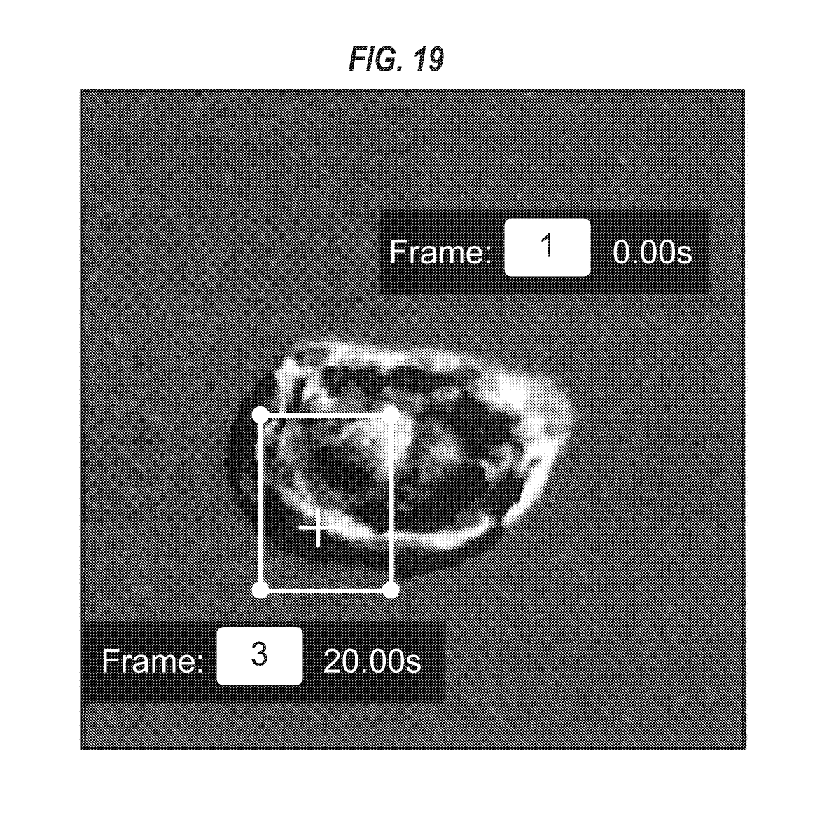

In correcting the DCE-MRI for patient movement, DCE-MRI frames in the temporal slices are aligned. If a patient moves during the examination, the joint inside the image frame changes its position as shown in FIG. 18, where four imaging frames with highly visible patient wrist motion are superimposed on top of each other. The outer boundaries of the wrist are outlined in red, indicated by reference “N”, to show the range of motion. FIG. 19 shows the result of ‘subtraction’ of frame 1 from frame 3 to demonstrate the range of movement.

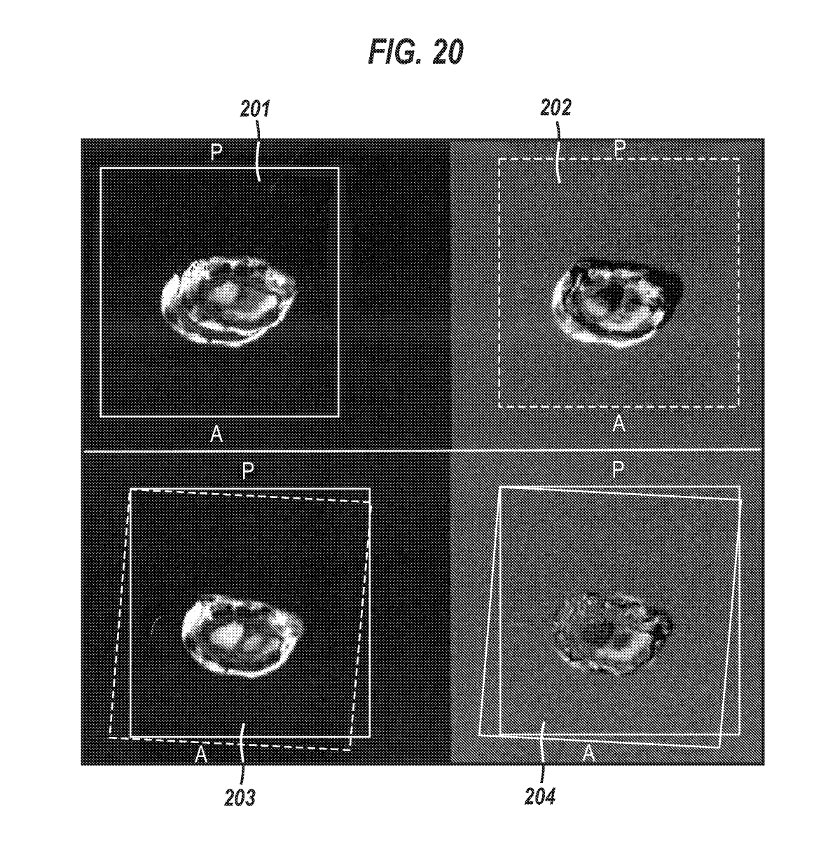

FIG. 20 illustrates motion correction. View 202 shows the superimposed DCE-MRI frames, view 201 shows the subtraction of the frames, view 204 shows the result of patient motion correction and the images within the frames are fully aligned while the image frame had to be moved and rotated, and view 203 shows the image subtraction after patient motion correction.

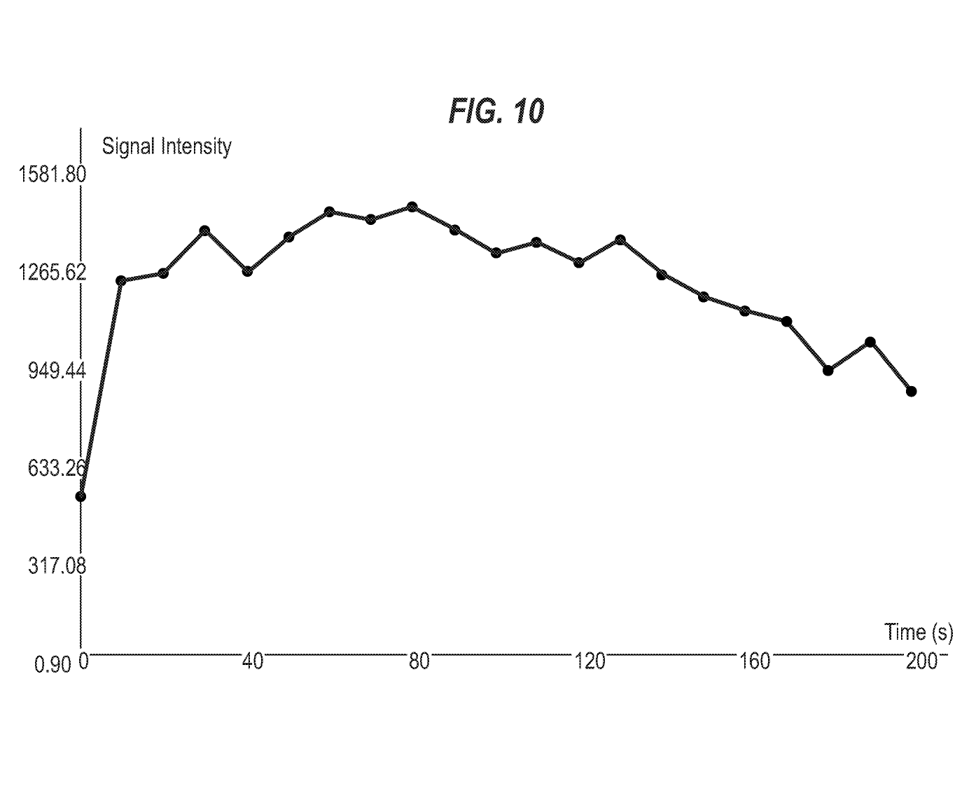

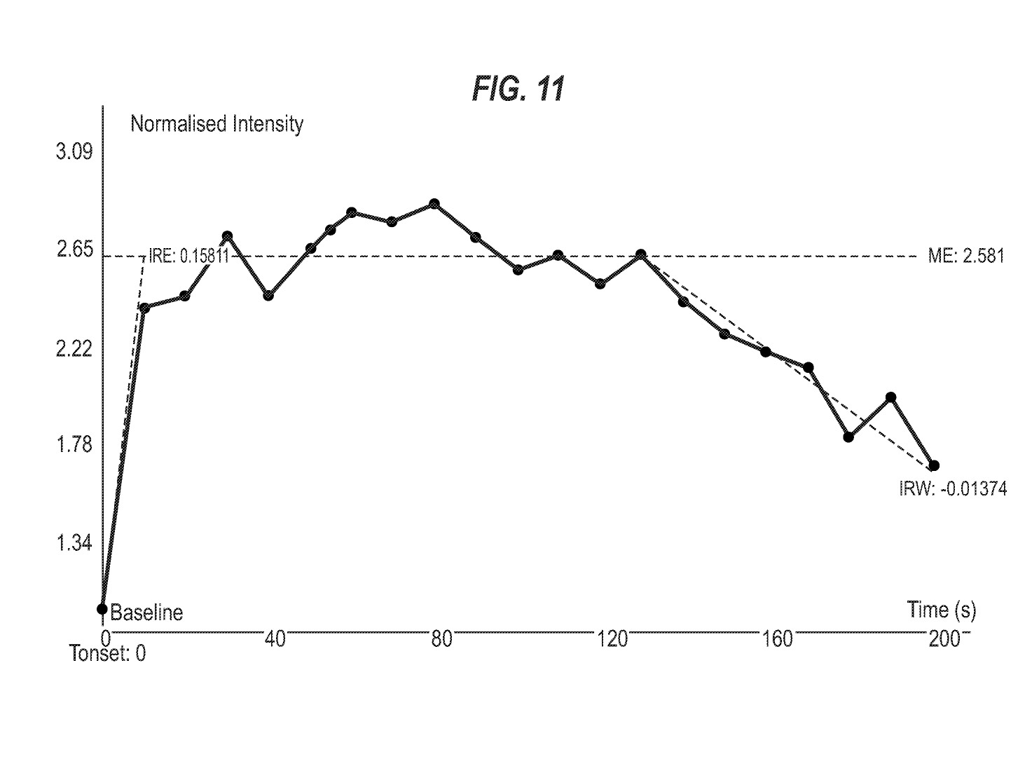

In normalizing the DCE-MRI signal intensity values, the normalization is done by subtracting the mean of the baseline intensity values (before the contrast uptake) from each signal intensity value, and then dividing the resulting values by the mean baseline value to express the signal intensity values in terms of multiples of the mean baseline value. For signal intensity versus time curves, this enforces the curve to start at 0. This helps reducing variability of intensity values in DICOM images obtained with various scanners. FIG. 10 shows a signal intensity graph and FIG. 11 shows a normalized version of the graph shown in FIG. 10. In the normalized graph, parameters of IRE, ME and IRW are marked showing contrast uptake, absorption and washout.

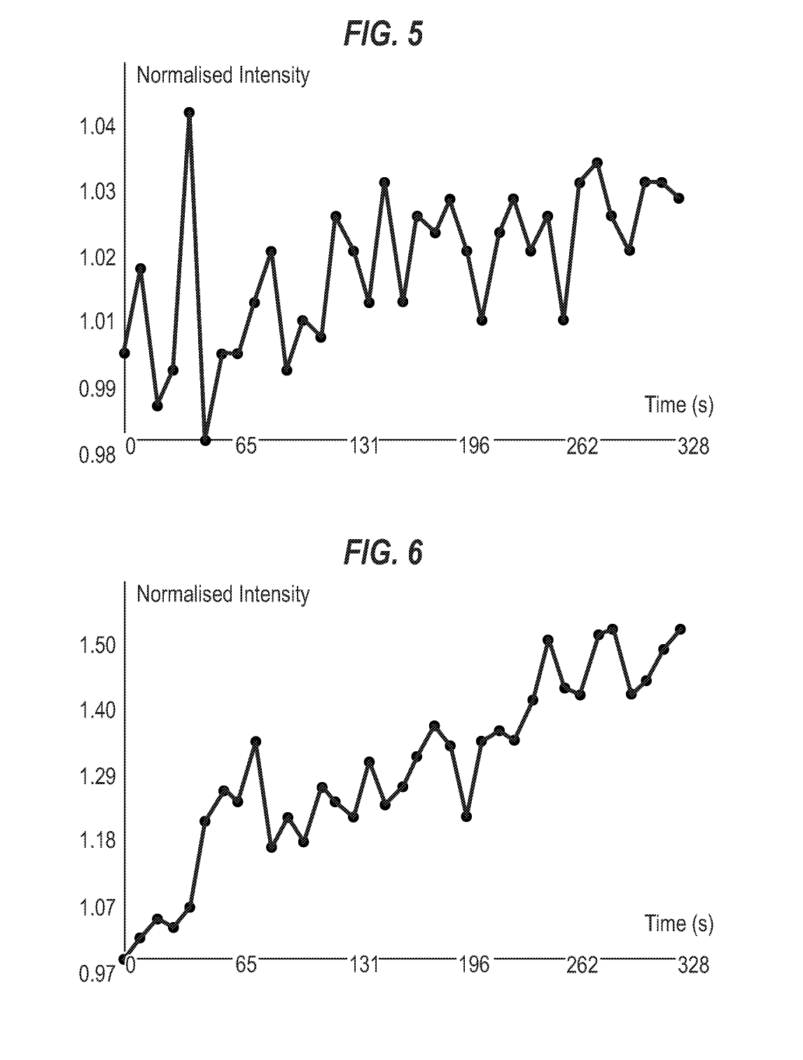

The signal intensity will vary for each pixel over time depending on the uptake of contrast agent for the tissue associated with the pixel. The signal intensity values are plotted to produce signal intensity (SI) versus time curve for each pixel. As can be seen from FIGS. 5 to 8, the signal intensity curves vary depending on the tissue associated with each pixel. All pixels can be classified into four distinctive types, each type being representative of a tissue type with a characteristic temporal pattern of enhancement:

### Type 0: No Enhancement

There is no enhancement in response to the contrast agent injection. As shown in FIG. 5, the SI curve shows just noise variations and corresponds to pixels located in the imaging marker, image background, bone interior or healthy non-inflamed tissue;

Type 1: Persistent enhancement

The SI curves exhibit baseline and wash-in phases, but do not reach an intensity plateau during the acquisition time interval, as shown in FIG. 6. Such SI curves normally correspond to pixels located in skin area, muscle or from artifacts;

### Type 2: Plateau Enhancement

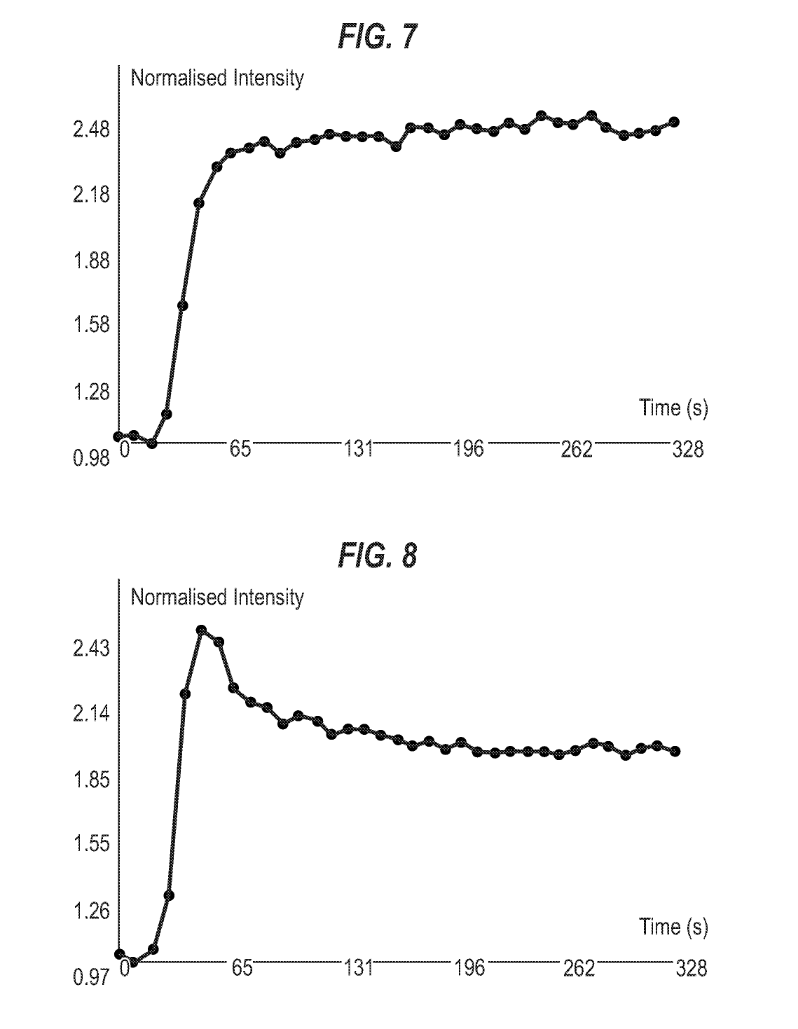

The SI curves clearly show the baseline, wash-in, and plateau phases, as shown in FIG. 7. The SI curves correspond to pixels located in vascular tissue. These tissues are normally located within inflamed synovitis, tenosynovitis, muscle, and oedema;

### Type 3: Wash-out Enhancement

The SI curves exhibit baseline, wash-in, plateau, and wash-out phases, as shown in FIG. 8. The SI curves correspond to pixels located normally located within severely inflamed synovitis and blood vessels.

The tissue type (0, 1, 2 or 3) of each pixel is identified through identifying the temporal pattern of contrast agent uptake for each pixel. Identifying the temporal pattern of contrast agent uptake for each pixel may comprise fitting a piecewise linear function to the signal intensity values. The function may comprise one or more of a wash-in phase, a plateau phase and a wash-out phase. Those pixels for which the best fit includes all three phases are classified as type 3, pixels for which the best fit includes only a wash-in phase and a plateau phase are classified as type 2, and pixels for which the best fit includes only the wash-in phase are type 1. Pixels which show no enhancement, wherein the best fit is a flat line, are type 0.

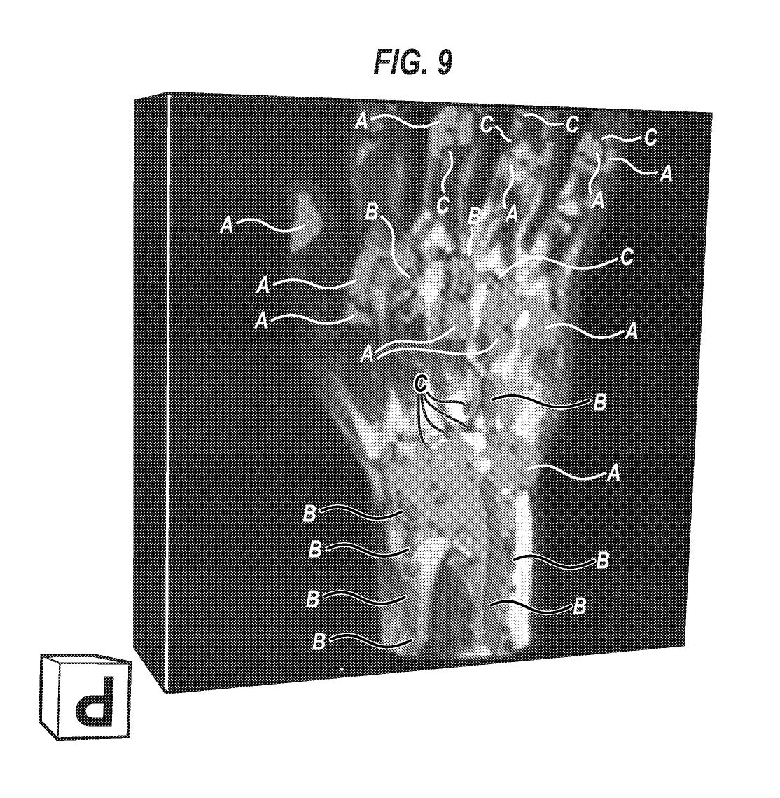

In selecting the region of interest, the pixels are colour coded depending on their tissue type, for example type 0 is no colour; type 1 is blue; type 2 is green; type 3 is red. These colours are superimposed on the pre-contrast DCE-MR image to form a map of tissue types. A user can view the map on a display to view the relative locations of the tissue types, as seen in FIG. 9, in which each voxel is colour coded according to the pattern of enhancement. Blood vessels are clearly shown in red, indicated by reference “B”, synovial tissue and oedema in green, indicated by reference “A” and some red, indicated by reference “B”; and tissues with no reaction to contrast agent have no colour.

A user can manually outline the region of interest, being guided by the colour map of the tissue types, and the computer software can automatically collect all enhancing pixels within the region of interest. The region of interest could be, for example, the entire image, the synovial lining or inside the bone.

Alternatively, the region of interest can be automatically selected by a processor operating on the data, for example by selecting a region which includes, for example, all of the pixels of tissue types 1, 2 and 3. Selecting a region may comprise identifying a region within the image which exhibits inflammation and which is larger than a minimum size. For example, selecting a region may comprise identifying a region with which has type 2 and/or type 3 pixels and wherein the region is larger than a minimum size. Multiple regions may be selected from the image, for example a blood vessel may be selected and at least one region of tissue inflammation may be selected. The blood vessel may be automatically selected through identifying a group of adjoining pixels which exhibit type 3 enhancement and which group is comparable in size to the size of a blood vessel.

In determining a value quantifying inflammation in the region of interest, the value is determined by a computer program analysing the signal intensity values which are associated with each pixel.

In one embodiment, shown in FIG. 2, in step 4A one or more values are determined which quantify the volume of inflammation in the region of interest.

The quantitative markers extracted by the computer program from the region of interest are one or more of:

N₁₃ total—total number of enhancing pixels in the area of interest, wherein N_total is the sum of N_persistent, N_plateau and N_washout;

N₁₃ persistent—total number of pixels with persistent pattern of enhancement in the area of interest;

N₁₃ plateau—total number of pixels with plateau pattern of enhancement in the area of interest; and

N₁₃ washout—total number of pixels with washout pattern of enhancement in the area of interest

The number of pixels for each of the above markers is normalized to the area of the joint/tissue or the physical size and reported either in ‘pixels’ or ‘square mm’. The region of interest may be a selected region of interest or it may be the entire image.

The value which quantifies inflammation is a function of one or more of N_total, N_persistent, N_plateau and N_washout. This value indicates the volume of inflamed tissue in the area of interest, and this value is output in step 5.

For example the determined and output value could be DEMRIS(Volume)

$$DEMRIS ( Volume ) = {\sum }_{area of interest} N\_plateau + N\_washout$$

DEMRIS(Volume) is a continuous measure and will be nearly 0 for healthy controls, patients in remission, and high for patients with inflammatory arthritis. Volumetric measurements of synovitis, oedema or tenosynovitis for healthy controls and patients in remission (N_total, N_persistent, N_plateau, N_washout) will be nearly at 0. A large volume of inflamed synovitis for patients with severe RA might be seen. DEMRIS(Volume) can also be referred to as DEMRIQ(Volume).

To further understand the pattern of the disease and to differentiate tissues on the basis of their vascularity and responsiveness to the contrast bolus, the height and slope of each signal intensity curve, approximated by liner segments, are measured. The following parameters are determined for each pixel from its signal intensity curve:

Maximum Enhancement (ME)—the average height of the contrast enhancement plateau for a pixel. ME is higher for the SI curves extracted from tissue with higher vascular perfusion;

Initial Rate of Enhancement (IRE)—steepness or slope of the SI curves during the wash-in phase. IRE is measured in units of percentage per second, with the highest values corresponding to the most vascular tissues;

Time of Onset of Enhancement (T₁₃ onset)—time when the contrast uptake begins. T_onset is measured in seconds and is the lowest for the tissue which start the update the earliest;

Initial Rate of Washout (IRW)—the slope of the SI curves during the wash-out phase. IRW is measured in units of percentage per second, and reflects at which rate the contrast is released by the tissue; and

Time of Washout (T₁₃ washout)—the time when the contrast washout begins. T_washout is measured in seconds.

The parameters IRE, ME, T_onset and IRW are indicated on the signal intensity versus time curve shown in FIG. 11.

For inter and intra examination comparison, all these parameters are extracted from the normalized SI curves.

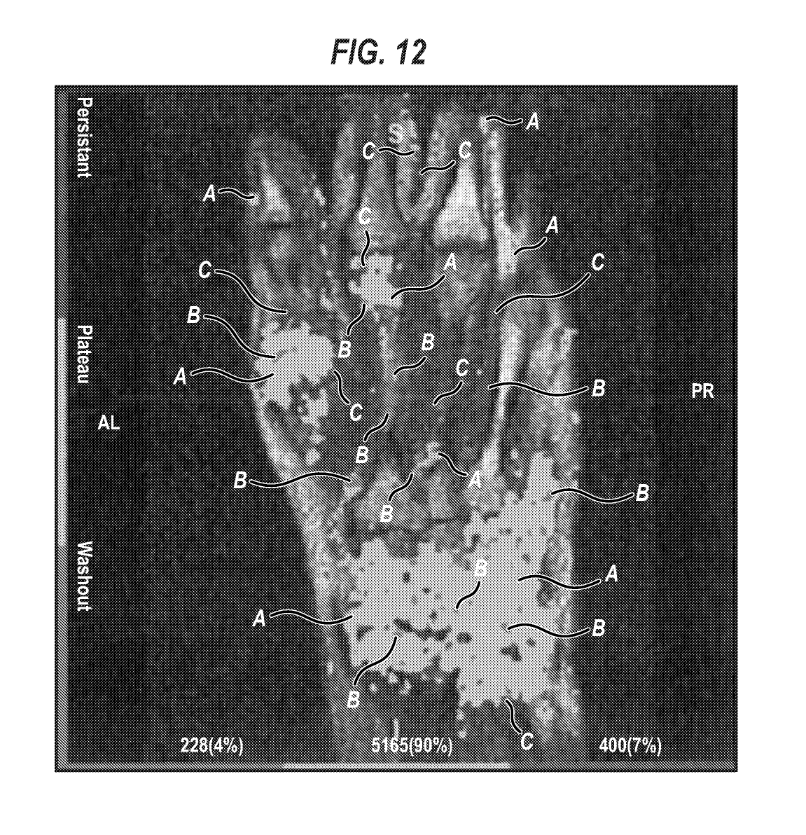

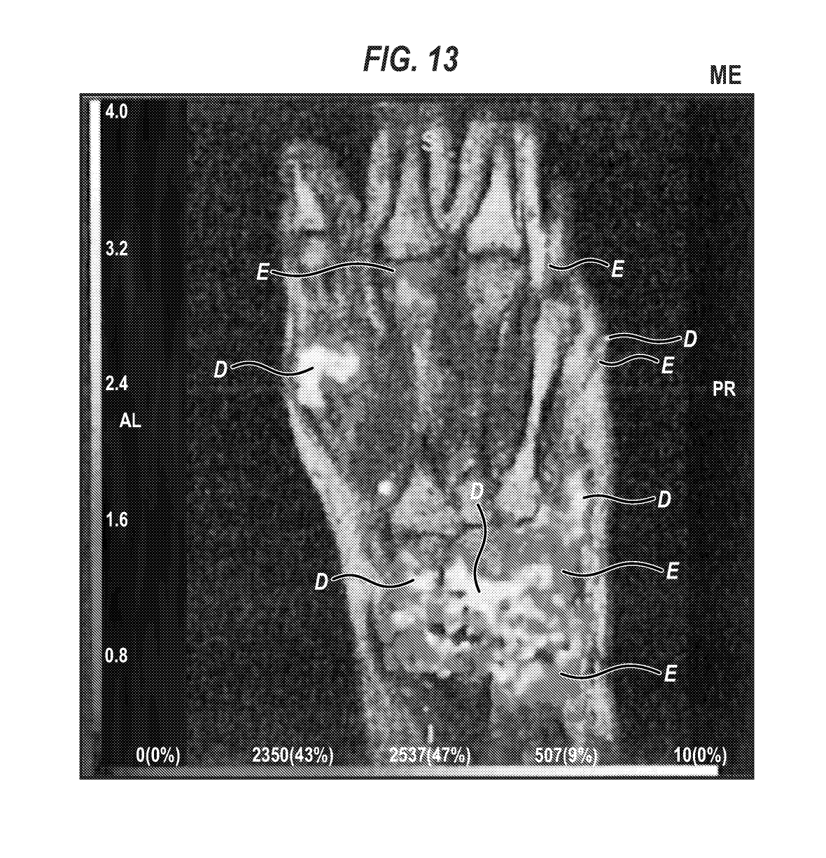

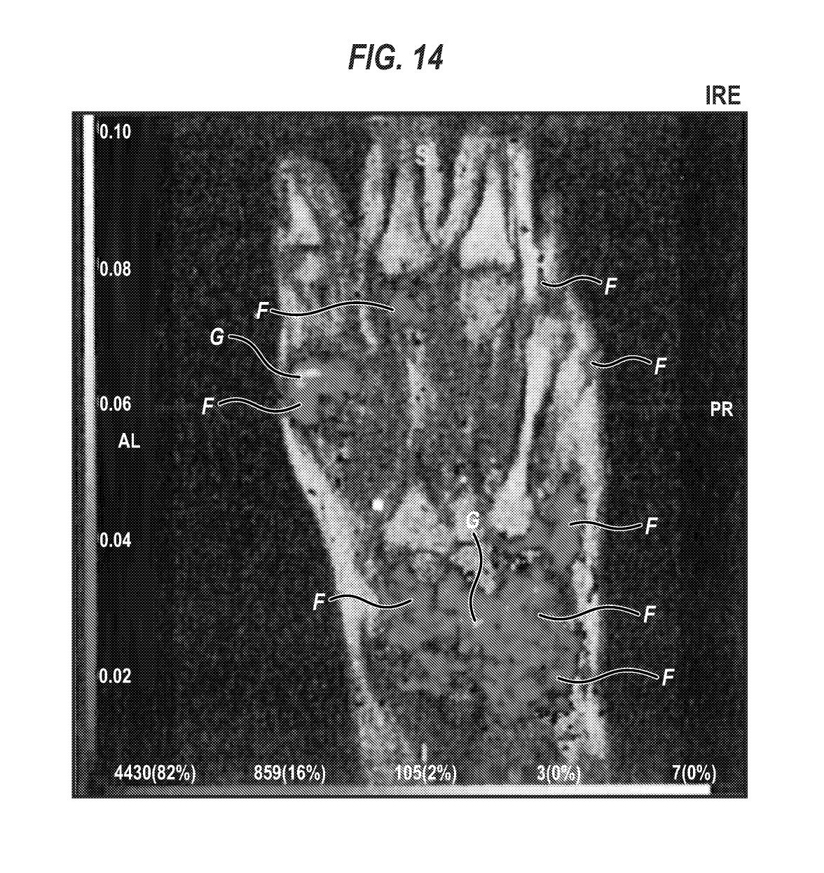

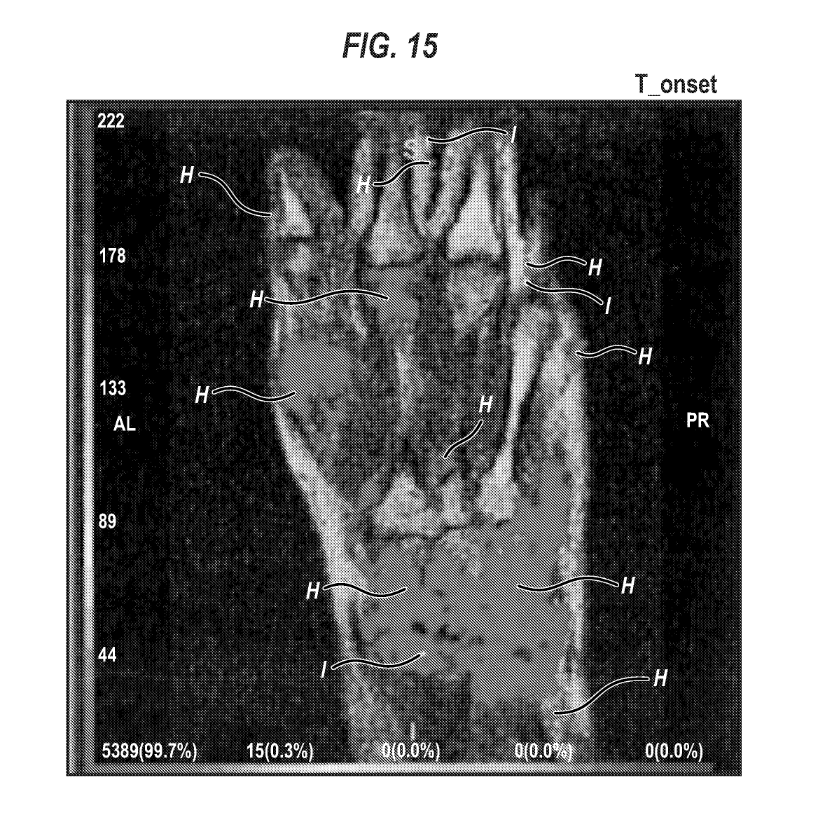

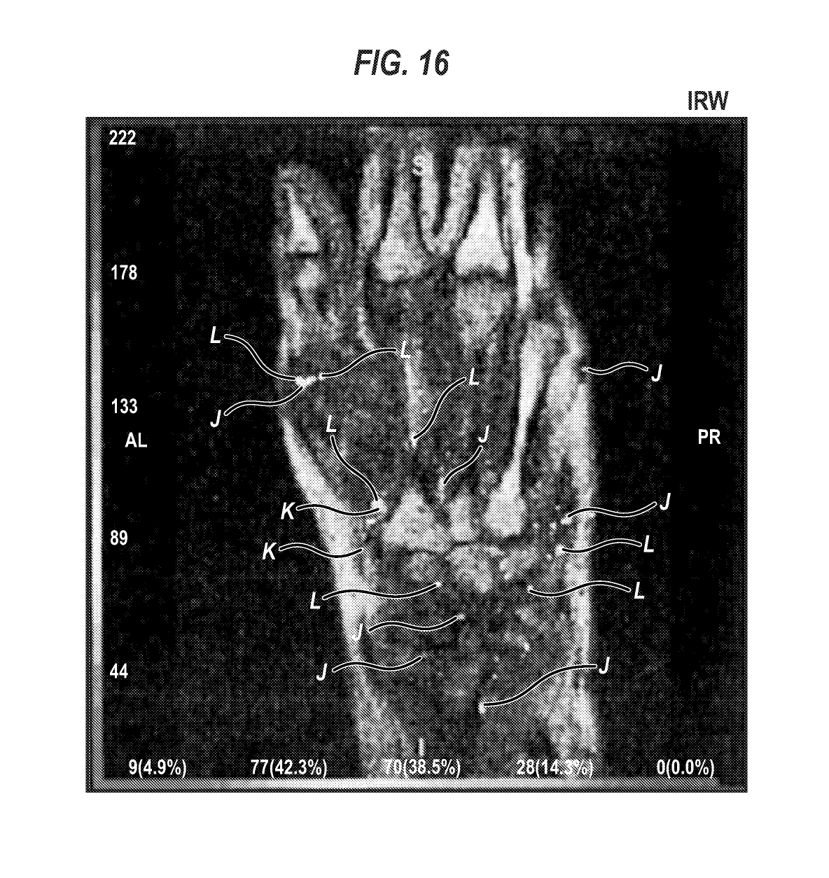

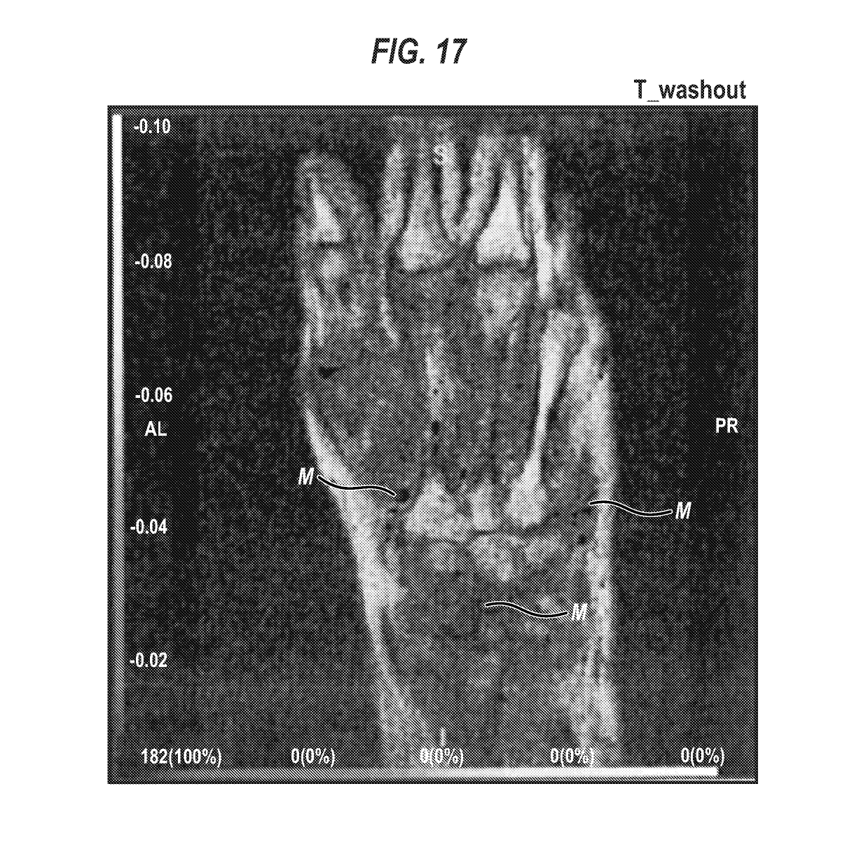

Maps of contrast uptake maximum enhancement, initial rate of enhancement, time of onset of enhancement, initial rate of washout and time of washout can be displayed on a display, as shown in FIGS. 12 to 17 respectively. Each map has colour bar, which visually guide the reader to the ‘hot spots’ of inflammation and a quantitative bar which shows the number of pixels enhancing up to a certain value. The maps can be used by the user to manually select the region of interest.

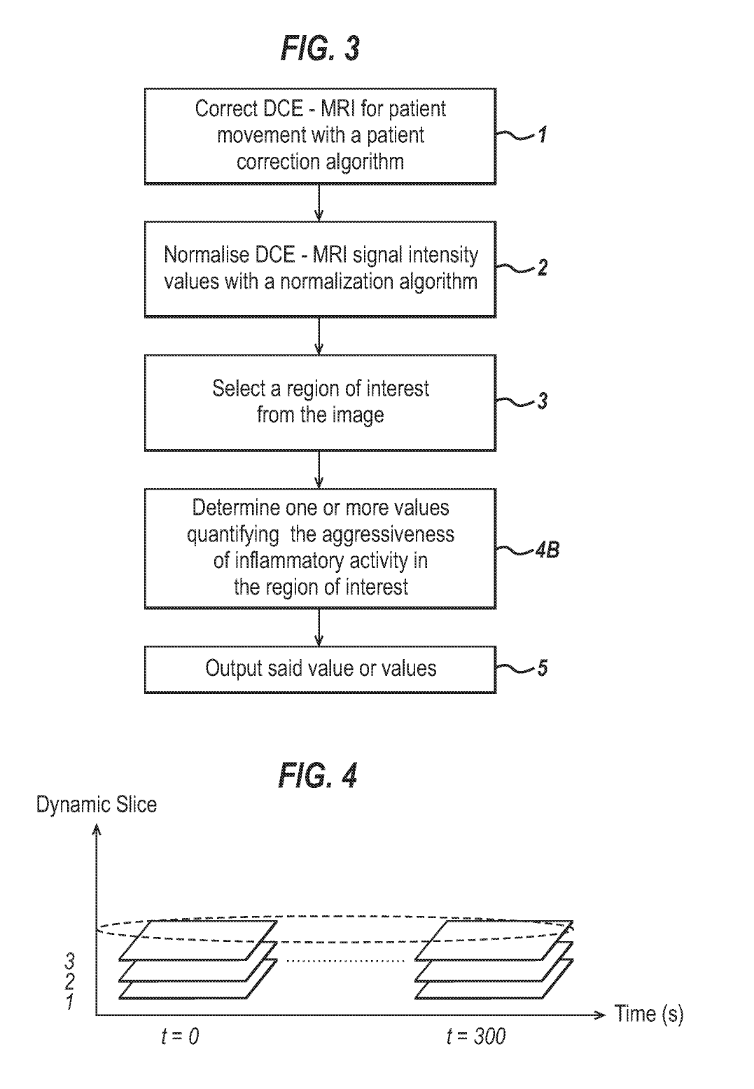

In the embodiment shown in FIG. 3, one or more values quantifying the aggressiveness of inflammatory activity in the region of interest are determined at step 4B. Said value or values are output in step 5.

For example, the determined and output value could be DEMRIS(Inflammation)

$$DEMRIS ( Inflammation ) = \frac{mean ( IRE inflamed area )}{mean ( IRE blood vessel )}$$

Where mean (IRE inflamed area) is the mean initial rate of enhancement in the inflamed area and mean (IRE blood vessel) is the mean initial rate of enhancement in a blood vessel.

Alternatively, DEMRIS (Inflammation) may be a combination of DEMRIS(Volume) multiplied by the mean initial rate of enhancement in the area of interest (mean (IRE)) or multiplied by the mean maximum enhancement in the area of interest (mean (ME))

$$DEMRIS ( Inflammation ) = mean ( IRE ) * {\sum }_{area of interest} Nplateau + Nwashout$$

$$or$$

$$DEMRIS ( Inflammation ) = mean ( ME ) * {\sum }_{area of interest} Nplateau + Nwashout$$

The area of interest may be the region of interest or the entire image.

DEMRIS(Inflammation) may also be referred to as DEMRIQ(Inflammation).

DEMRIS(Inflammation) is a continuous measure in the range from [0 . . . 1]. It is close to zero for controls and patients in remission and close to 1 for the patients with severe RA.

Other combinations of the parameters N_total, N_persistent, N_plateau, N_washout ME, IRE, Tonset, IRW, Twashout, may be determined and output as a value quantifying inflammation. For example, the value may be a function of:
N_plateau×mean(ME);
N_plateau×mean(IRE); or
N_total×mean(IRE)

Where mean(ME) is the mean maximum enhancement for pixels in the region of interest, and mean(IRE) is the mean initial rate of enhancement for pixels in the region of interest.

The computer outputs more than one output value which quantifies inflammation in the tissue. At least one of said values indicates the volume of inflamed tissue in the region of interest and at least one of said values indicates the severity of inflammation. Said values are continuous score values residing in a range of continuous score values quantifying the inflammation.

In a preferred embodiment, both DEMRIS(Volume) and DEMRIS(Inflammation) are determined and output to a user.

In a further preferred embodiment, the output comprises data with continuous volumetric measurements of inflammation and the inflammation aggressiveness measures. The data comprises N_total, N_persistent, N_plateau, and N_washout, and the mean and standard deviation of ME, IRE, T_onset, IRW, and T₁₃ washout. The data is preferably output in the form of a table.

Table 1 below illustrates the algorithm steps and related computer support in an embodiment.

TABLE 1

| Algorithm Steps | | Computer Support |
|---|---|---|
| 1. | Correct DCE-MRI exam for patient | Patient Motion Correction |
| | movement | algorithm |
| 2. | Normalize DCE-MRI SI curves to a | Automated adjustment to |
| | baseline including subtracting the mean | the baseline algorithm: |
| | values of pre-contrast frames from all | SI normalized = SI |
| | other planes. This ensures that SI | original − b, b—baseline |
| | curves are consistent and start at 0; | |
| 3. | Using parametric maps of contrast | Computer-guided ROI |
| | uptake, ME, IRW or IRE and T_onset | placement |
| | or T_washout, place a rough region of | |
| | interest around the inflamed areas and | |
| | region of interest around the vessel | |
| 4. | Record the mean measurements of IRE | Automated measurement of |
| | for the inflamed ROI and mean IRE for | DEMRIS(Inflammation) |
| | the blood vessel ROI. | |
| 5. | Record the total Volume of | Automated measurements |
| | Inflammation in either 1 slice or all | of DEMRIS(Volume) |
| | slices as a sum of pixels with | |
| | N_plateau and N_washout | |

Note that step 1, motion correction, is not always necessary. It may be possible to immobilize patients in the scanner,

The output values are correlated with known parameters so that the relationship between the output values and other disease markers is established. The output values are correlated with patient classifications to allow clinicians to relate the output values to known classifications, for example a value of DEMRIS(Inflammation) in the range of 0.06 to 0.2 may correspond to mild RA.

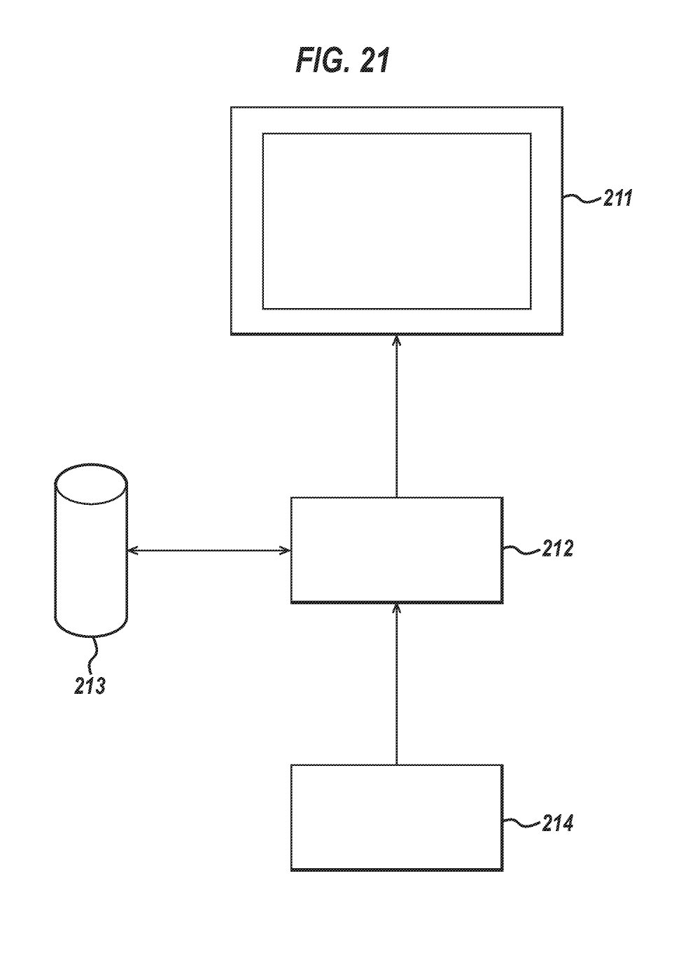

FIG. 21 illustrates a system for performing the method described herein. The system has a processor 212 in communication with a storage device 213. The computer program executable to perform the method described herein is stored on the storage device 213. An input device 214, for example a computer pointing device, is in communication with the processor 212. User input is via the input device 214. A display 211 is in communication with the processor 212. Output of the processor can be displayed on the display 211.

### Studies

In healthy controls or patient in remission, we expect dynamic parameters of inflammation, ME and IRE and volumetric measures such as N_plateau and N_washout to be close to 0 and not to vary in response to the treatment and over time. An analysis of sensitivity and stability of dynamic parameters in healthy controls confirm that the automatically obtained parameters are sensitive and stable over 12 months time to the 0.05% error level which is attributable to imaging artefacts and/or hardware instability at imaging.

In a study, SI curves were extracted from the wrist joint of a healthy controls and the blood vessel; DCE-MRI on 3T Philips, TR/TE/FA: 3.8 ms/2.1 ms/20°, FOV: 120×95×80 mm³, Acquisition matrix: 96×75, 127 temporal slices, 40 dynamic frames in 3D scan mode, voxel size: 1.25×1.25×0.63 mm, dynamic time: 10.3 sec, time: 6 min 52.8 sec. 40 sec delay from the start of image acquisition to contrast injection of Gadolinium-DTPA, 0.2 ml/kg. For this healthy volunteer, DEMRIS(Volume) here is 2 pixels and DEMRIS(Inflammation) is 0.

An extensive study on healthy volunteers conducted longitudinally over 1 year where a dominant hand of 10 healthy volunteers (3♂ and 7♀, age range: 24-40 years, BMI 19-29.9 kg/m2) were imaged with 3T Philips MRI at baseline, week 12, 24 and 52, demonstrated that the inherent variability in a new automated quantitative DCE-MRI methodology, Dynamic Contrast Enhanced MRI Scoring (DEMRIS) is small and remains stable throughout the year in healthy subjects, and correlates well with RAMRIS. DEMRIS is also referred to as DEMRIQ which stands for Dynamic contrast Enhanced MRI Quantification. Further, this study compared DEMRIS in healthy volunteers with DEMRIS in RA patients and confirmed the suitability of DEMRIS as DCE-MRI quantitative method for use in longitudinal studies of inflammatory arthritis. DEMRIS delivers a convenient approach to the extraction of heuristics and parametric maps permits easy visual assessment of the degree of inflammation in RA patients, which allows a more accurate analysis of the extent of the disease and differentiation of various tissues as well as more reliable separation of healthy subjects from active patients.

A similar study was performed using low field scanners where two centres applied DEMRIS for analysis of 135 active RA patients and 5 healthy controls using a 0.2 T musculoskeletal dedicated extremity scanner (C-scan and E-scan respectively, Esaote Biomedica, Genoa, Italy). The patients had Ultrasound, conventional MRI, DCE-MRI in addition to measuring CRP, early morning stiffness, DAS28 and DAS44 and other clinical and preclinical markers. DCE-MRI was performed following the Gd-DTPA injection (0.2 mmol/kg of body weight), resulting in 22-30 consecutive fast SE (TR/TE 100/16, FOV/imaging matrix 150 150/160 128), or GRE images (TR/TE 60/6, FOV/imaging matrix 160 160mm/256 128) in three pre-positioned planes every 10-15 s. Slice thickness was 4 mm in the coronal plane or 5 mm in the axial plane; the total scanning time was 300 s.

The study showed that DEMRIS(Volume) for healthy controls was close to 0, whereas the patients scored high on DEMRIS(Volume), which ranged from 10% to 50% of the entire joint volume depending on the disease stage. DEMRIS(Inflammation) was 0 to 0.05 in healthy controls and significantly higher for patients. For healthy controls on average the number of enhancement pixels is less than 0.5-1% of the joints' volume; for active patients, it might reach up to 50% of the joints' volume. In this study, all quantitative scores were extracted from the maps in a fully automated manner and were used to objectively differentiate health controls from patients with active RA. Later studies deployed computer guided ROI methods to roughly outline joints and avoid blood vessels, which further increased sensitivity and responsiveness of the method.

A study on the Reliability and responsiveness of DEMRIS in patients with active RA focused on the analysis of active RA patients and their responsiveness to treatment. DCE-MRI was performed in 12 clinically active RA knee joints before and 1, 7, 30, and 180 days after intra-articular injection with 80 mg methylprednisolone. All patients were scored with DEMRIS, which allowed achieving very high intra- and inter-reader ICCs, 0.96-1.00. The study also demonstrated high responsiveness with a standardized response mean of up to 2 for the DEMRIS volume and inflammation reduction in patients following the treatment.

A further study evaluated DEMRIS in regions of interest separately placed in oedema and synovium and compared dynamic parameters with clinical assessment, Ultrasound Doppler scores and RAMRIS. It was concluded, that DEMRIS of synovitis and bone marrow oedema correlate strongly with RAMRIS measures of synovitis and oedema. 36 RA patients had a routine 3T MRI examination (Siemens, Verio®) of the most symptomatic hand. DCE-MRI was performed in 18 slices every 9 seconds, with 30 repetitions covering the whole hand, started at the time of IV contrast injection (Prohance 0.1 mmol/kg). Correlation between the RAMRIS scores and the DEMRIS(Volume) in the wrist joint were r=0.84 for BMO and r=0.83 for synovitis with p<0.001; for the MCP joints r=0.73 for BMO and r=0.80 for synovitis p<0.001, respectively. Correlation between DEMRIS(Inflammation) and RAMRIS in synovium wrist was r=0.83 and bone marrow oedema wrist r=0.69 ; for synovim MCP r=0.85, and BMO MCP r=0.58, p<0.001.

The study on discrimination of early rheumatoid arthritis patients and healthy persons by conventional and dynamic contrast-enhanced MRI, applied DEMRIS and RAMRIS to score RA in the hand of 14 early RA patients and 26 healthy persons using a 1.0 T Siemens Impact MRI unit (Siemens, Erlangen, Germany) at baseline, and post 6 and 12 months of DMARD treatment. The study showed that DEMRIS(Volume) and DEMRIS(Inflammation) are sensitive measures for separating healthy controls and patients with early RA. Statistical analysis of the dynamic MRI parameters extracted from the data acquired from healthy persons and early RA patients, have shown that there is significant quantitative difference in the amount of inflammation: the median value of DEMRIS(Volume) was 3 and 362 for healthy persons and patients, respectively; DEMRIS(Inflammation) was significantly higher in active patients, p≦0.003, proving its sensitivity to change. Further case reports and smaller studies were performed to show applicability of quantitative DEMRIS in monitoring patient response in early RA and PsA. These studies conclude that this scoring methodology is highly sensitive for monitoring the early inflammatory treatment response in patients and enables precise quantitative and reliable measurements of the patient progress comparing to Ultrasound, semi-automated MRI scoring methods, and clinical assessment.

In a group of healthy controls and RA patients with wide spectrum of clinical and imaging disease activity we found a high correlation between the proposed markers DEMRIS(Inflammation) and DEMRIS(Volume) and RAMRIS scores of synovitis and BME. These results support the established definition of synovitis and BME as volume of inflamed synovium (synovitis) and volume of osteitis (BME) in the various bones, respectively. However, the new scoring methodology, allows for continuous, threshold independent analysis of patient response to treatment as well as very early detection of the disease.

1. A computer-implemented method for quantifying inflammation in tissue or anatomy, comprising:
acquiring image data pertaining to at least one image of tissue or anatomy;
analysing the image data comprising determining a first value quantifying inflammation in the tissue or anatomy; and
outputting said first value,
wherein the first value is determined to be a continuous score value residing in a range of continuous score values quantifying the inflammation.

2. A method according to claim 1, wherein the step of analysing comprises determining the first value based on a continuous function being applied to the image data.

3. A method according to claim 2, wherein the first value quantifies the volume of inflammation.

4. A method of claim 3, wherein the step of analysing the image data further comprises determining the first value by quantifying the volume of inflammatory activity in the tissue or anatomy.

5. A method of claim 4, wherein the step of quantifying the volume of inflammatory activity comprises quantifying based on a first continuous function being applied to the image data.

6. A method according to claim 1, wherein the first value quantifies the aggressiveness of inflammatory activity.

7. A method of claim 6, wherein the step of analysing the image data further comprises determining the first value by quantifying the aggressiveness of inflammatory activity in the tissue or anatomy.

8. A method of claim 7, wherein the step of quantifying the aggressiveness comprises quantifying based on a second continuous function being applied to the image data.

9. A method according to claim 1, wherein each image is a magnetic resonance image (MRI).

10. A method according to claim 9, wherein the image is a plurality of temporal magnetic resonance images.

11. A method according to claim 1, wherein each image is a computed axial tomography image or an ultrasound image.

12. A method according to claim 1, wherein the tissue has been exposed to a contrast agent.

13. A method according to claim 12, wherein analysing the data comprises analysing a temporal pattern of contrast agent uptake.

14. A method according to claim 1, further comprising identifying a region of interest of the tissue and selectively analysing data pertaining to the image of the tissue or anatomy in the region of interest.

15. A method according to claim 1, wherein the analysed data comprises signal intensity values for one or more pixels of the image at one or more time points.

16. A method according to claim 1, wherein analysing the data comprises classifying one or more pixels of the image into groups, each group representative of a tissue type.

17. A method according to claim 16, wherein analysing the data comprises analysing a temporal pattern of contrast agent uptake for said one or more pixels of the image for determining the tissue type of each pixel.

18. A method according to claim 16, wherein the step of analysing the data comprises identifying one or more pixels of the image of a first tissue type.

19. A method according to claim 18, wherein the step of analysing the data comprises summing the number of pixels of the image determined to be of the first tissue type.

20. A method according to claim 18, wherein the step of analysing the data comprises identifying one or more pixels of the image of a second tissue type.

21. A method according to claim 20, wherein the step of analysing the data comprises summing the number of pixels of the image determined to be of the second tissue type.

22. A method according to claim 18, wherein analysing the data comprises normalizing the number of pixels of the first tissue type and/or of the second tissue type.

23. A method according to claim 22, wherein the normalization is based on the dimensions of the imaged tissue, or number of pixels in the image as a whole.

24. A method according to claim 18, wherein the first value is a function of the total number of pixels of the first tissue type and/or the second tissue type.

25. A method according to claim 24, wherein the first value is a function of the normalized number of pixels of the first tissue type and/or of the normalized number of pixels of the second tissue type.

26. A method according to claim 18, wherein the first tissue type is tissue identified as having a plateau enhancement.

27. A method according to claim 18, wherein the first tissue type is tissue identified as having a wash-out enhancement.

28. A method according to claim 27, wherein the second tissue type is tissue identified as having a plateau enhancement.

29. A method according to claim 1, further comprising generating display data for display pertaining to a parametric map, preferably a colour-coded parametric map, for an observer to visualise locations of different tissue types.

30. A method according to claim 29, further comprising selecting a region of interest of the tissue based on input from the observer.

31. A method according to claim 1, wherein analysing the data comprises determining an initial rate of enhancement in an inflamed area.

32. A method according to claim 31, wherein analysing the data further comprises determining an initial rate of enhancement in a blood vessel.

33. A method according to claim 31, wherein the first value is a function of the initial rate of enhancement in the inflamed area.

34. A method according to claim 33, wherein the output value is a function of the initial rate of enhancement in the inflamed area and the initial rate of enhancement in the blood vessel.

35. A method according to claim 34, wherein the output value is a function of the ratio of the initial rate of enhancement in the inflamed area and the initial rate of enhancement in the blood vessel.

36. A method according to claim 31, wherein analysing the data comprises determining a mean initial rate of enhancement in an inflamed area.

37. A method according to claim 31, wherein analysing the data comprises determining a mean initial rate of enhancement in the blood vessel.

38. A method according to claim 37, wherein the output value is a function of the ratio of the mean initial rate of enhancement in the inflamed area and the mean initial rate of enhancement in the blood vessel.

39. A method according to claim 31, wherein analysing the initial rate of enhancement in the inflamed area and/or in the blood vessel comprises measuring the slope of signal intensity curves for one or more pixels of the image.

40. A method according to claim 39, wherein analysing the data comprises approximating the slope of each curve by a linear segment.

41. A method according to claim 1, further comprising correcting the images for patient movement.

42. A method according to claim 39, wherein analysing the data comprises normalizing the signal intensity curves of one or more pixels of the image to a baseline, preferably by subtracting the mean values of pre-contrast frames from all other planes.

43. A method according to claim 1, wherein the first value is any real value in a continuous range of values between 0 and 1; 0 and 5; 0 and 10; 0 and 100; or 0 and 1000.

44. A method according to claim 1 for quantifying inflammation in tissue or anatomy of patients with inflammatory arthritis.

45. A method according to claim 1 for quantifying inflammation in tissue or anatomy of patients with cancer, in particular breast cancer or brain cancer.

46. A method according to claim 1, wherein outputting said first value comprises conveying said first value to a user.

47. A computer program comprising executable instructions for execution on a computer, wherein the executable instructions are executable to perform the method of claim 1.

48. An apparatus for quantifying inflammation in tissue, comprising:
a memory, wherein the memory comprises a computer program comprising executable instructions for execution by a processor to perform the method of claim 1; and
the processor.

49. An apparatus configured to perform the method of claim 1.
